(19) United States
(12) Patent Application Publication
(10) Pub. No.: US 2020/0297284 A1
(43) Pub. Date: Sep. 24, 2020

(54) CARDIAC SCAR DETECTION

(71) Applicants: Siemens Healthcare Limited, Camberley (GB); King's College London, London (GB)

(72) Inventors: Hugh O'Brien, London (GB); Steven Niederer, London (GB); Peter Mountney, Camberley (GB)

(73) Assignees: Siemens Healthcare Limited, Camberley (GB); King's College London, London (GB)

(21) Appl. No.: 16/791,095

(22) Filed: Feb. 14, 2020

(30) Foreign Application Priority Data
Mar. 20, 2019 (GB) 1903838.9

(51) Int. Cl. A61B 5/00; A61B 5/055; A61B 6/03; A61B 6/00; G06N 3/08; G06N 3/04; G16H 30/40; G16H 50/50; G06K 9/62; G06T 11/00; G01R 33/56; G01R 33/48

(52) U.S. Cl. CPC A61B 5/7267; G06T 2211/424; A61B 5/055; A61B 5/0035; A61B 6/032; A61B 6/503; A61B 6/5247; G06N 3/08; G06N 3/04; G16H 30/40; G16H 50/50; G06K 9/6257; G06T 11/005; G01R 33/5608; G01R 33/4812; A61B 2576/023; G06K 2209/051; A61B 5/0044

(57) ABSTRACT

Techniques are disclosed related to using anatomical mask data acquired via magnetic resonance imaging (MRI) scans to train a convolutional neural network (CNN). The training may include the verification of cardiac scar tissue locations data obtained from the anatomical mask data with a reliable system for doing so, such as ground truth data from enhanced cardiac MRI late gadolinium enhanced (LGE) scans. Once the CNN is adequately trained using the anatomical mask data, the CNN may be used to identify cardiac scar tissue from image data obtained from medical imaging modalities other than MRI.

### CROSS-REFERENCE TO RELATED APPLICATIONS

The present application claims the benefit of the filing date of Great Britain patent application no. 1903838.9, filed on Mar. 20, 2019, the contents of which are incorporated herein by reference in their entirety.

### TECHNICAL FIELD

The present disclosure concerns techniques of scar detection and, in particular, to techniques using data acquired via contrast enhanced MRI magnetic resonance imaging (MRI) scans to produce a scar detection network using machine learning techniques.

### BACKGROUND

Cardiac scar detection is important for many clinical applications. The location of scar has been shown to be useful in planning for implant procedures, such as for Cardiac Resynchronization Therapy (CRT) and pacemakers. It is also known to be beneficial for interventions including revascularization. Avoiding scar tissue in the placement of CRT leads, for instance, has been linked to better outcomes, as pacing scar does not have the desired effect due to differences in tissue conductivity. Revascularizing tissue after a heart attack that has already become scarred has also been shown to not produce improved outcomes.

Cardiac magnetic resonance imaging (MRI) with late gadolinium enhancement is the current clinical gold standard for cardiac scar detection. Computed tomography (CT) could be an ideal modality for this task, as modern CT provides much higher resolution than enhanced MRI, both spatially and temporally. CT is also a common first imaging method for cardiac patients. Furthermore, MRI is contraindicated in many patients due to renal problems such as kidney disease, which renders the contrast agent too dangerous. See Reference [3]. MRI may also be contraindicated due to existing implants, which may cause large image artifacts. As discussed in the references [1] and [2], delayed enhancement methods for CT using iodine-based contrast agents are available for CT, but are not in wide clinical use. Without enhancement, there is no method of differentiating cardiac muscle tissue and scar tissue using CT image intensities alone.

### SUMMARY

As noted above, the high spatial and temporal resolution of CT scans mean there are several advantages to using it as a preoperative planning modality for cardiac applications. However, there is still a need to develop a robust scar detection method using this data to avoid having to also perform an enhanced MRI.

Currently, the gold standard for cardiac scar imaging is MRI using late gadolinium enhancement. See reference [4]. However, this requires an injection of a gadolinium contrast agent, and for MRI not to be contraindicated for the patient. Scar tissue is also detectable using other modalities. For instance, positron emission tomography (PET) and single-photon emission computerized tomography (SPECT) scans have been used by taking the uptake in tracers as an indication of healthy tissue. While in active clinical use, however, these are low resolution and have been shown to not be as accurate as MRI. See reference [5].

There are, however, biomarkers indicative of scar tissue. For instance, some studies have used scar estimation thickness measurements of the heart wall tissue to identify cardiac scar tissue, as wall thinning has been shown to be related to the presence of scar tissue. See reference [6]. However, such methods require an explicit cut-off point, i.e. a defined threshold wall thickness, to be established to indicate scar tissue, and the use of such a threshold value is not easily generalizable to all patient populations.

While CT cannot detect scar tissue from the differences in pixel intensity alone, it does image the anatomy with a very high resolution. Moreover, there are known surrogates for scar tissue that can be extracted from the anatomy alone to produce a scar estimate, and this is not dependent on intensity in the images yielded by enhancement via contrast agents. Wall thinning, for instance, has been used as an early method of predicting scar tissue using echo, before contrast agents and MRI became more widely available. See reference [7]. Further, other markers, such as subtle changes in the heart wall shape, may also be indicative of scar presence.

Therefore, the embodiments described in the present disclosure address the current shortcomings of scar tissue identification by leveraging the use of existing automated segmentation tools to construct an abstract image mask of the cardiac anatomy showing endocardium and epicardium walls. In particular, the abstract image mask shows the cardiac wall thickness and shape, which may be extracted using multiple imaging modalities. Then, using data from contrast-enhanced MRI scans, multiple abstract image masks may be extracted and used as training data as a model input, which can be trained by verifying the output of the model, which attempts to identify the location and quantity of scar tissue using the abstract image masks as the model with results from a known reliable standard (e.g. LGE) to produce a scar detection network using machine learning techniques.

As an example, and further discussed below, a method in accordance with an embodiment of the present disclosure includes extracting anatomical mask training data from MRI scans, which includes, in the example of cardiac scar detection, extracting a set of left ventricle wall masks as a result of multiple imaging slices obtained (e.g., via cardiac MRI scans) for each patient in a patient “training pool.” Thus, continuing this example, each one of the set of left ventricle wall masks includes a plurality of slices extracted from each one of a set of different patients in the training pool. In other words, multiple masks are obtained at different parts of the anatomy as part of the anatomical mask extraction process, and in the aggregate this collection of different masks from different patients represents the anatomical mask training data. The extracted anatomical masks may then be used as a model input to train a convolutional neural network (CNN). Using anatomical mask training data obtained in this manner advantageously allows for the use of alternatives to LGE scans in the event that such a scan is not possible.

The training scan or processing pipeline, therefore, results in the extraction of anatomical mask training data, which may be used as a model input to the CNN that attempts to identify a location and quantity of cardiac scar tissue. In an aspect, the CNN may be trained using the anatomical mask data to detect scar tissue from acquired scan images, and to infer the presence and location of scar tissue based on the anatomical mask training data, which is verified with scar data, or the result of a reliable imaging scan, such as an enhanced contrast MRI scan, for example. Another processing pipeline (e.g. non-MRI imaging modality such as CT) may then automatically segment a mesh from imaging data (e.g. CT imaging data) and slice the mesh to produce the same mask format as the anatomical mask training data that was used to train the CNN model. With correct scaling and mask production, the model can predict scar tissue using MRI or non-MRI imaging modalities. In other words, and as further discussed below, other imaging modalities in which meshes can be generated can work in accordance with their own processing pipelines using the same model.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 illustrates a representation of a magnetic resonance device, in accordance with an exemplary embodiment of the present disclosure.

FIG. 2 illustrates a scar classification system using a convolutional neural network, in accordance with an exemplary embodiment of the present disclosure.

FIG. 3 is an example flow, in accordance with an exemplary embodiment of the present disclosure.

The exemplary embodiments of the present disclosure will be described with reference to the accompanying drawings. The drawing in which an element first appears is typically indicated by the leftmost digit(s) in the corresponding reference number.

### DETAILED DESCRIPTION

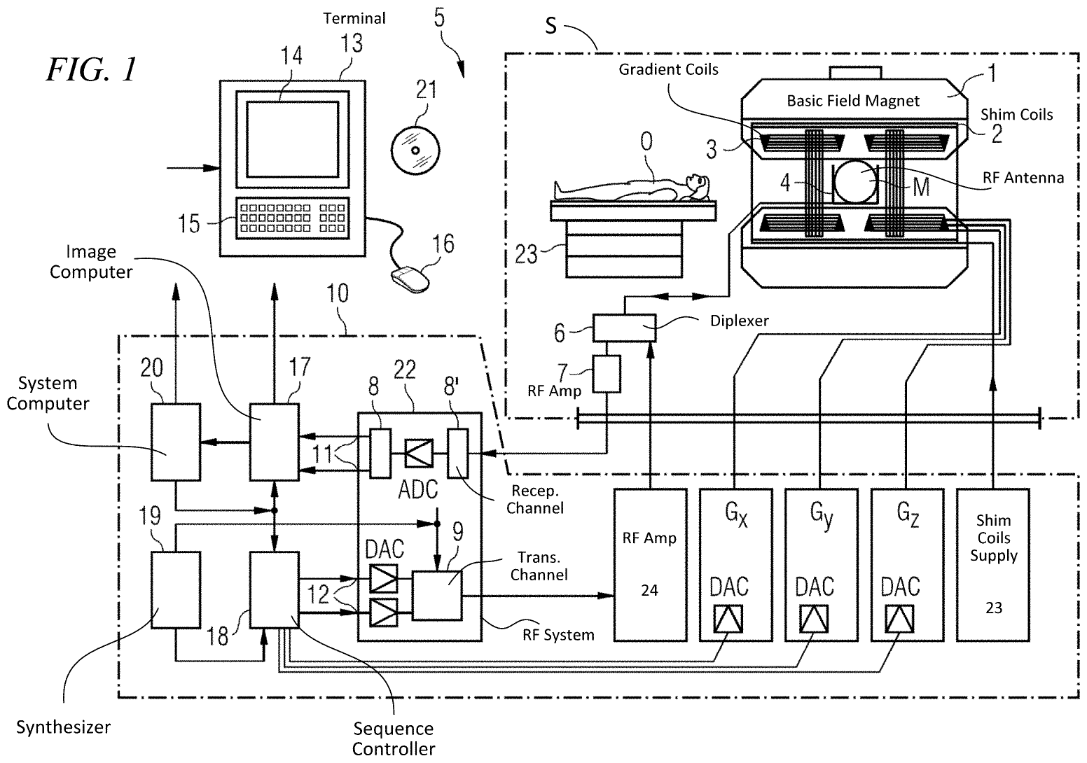

FIG. 1 illustrates a representation of a magnetic resonance device, in accordance with an exemplary embodiment of the present disclosure. As shown in FIG. 1, a magnetic resonance apparatus 5 (e.g., a magnetic resonance imaging or tomography device) is shown. A basic field magnet 1 generates a temporally-constant strong magnetic field for the polarization or alignment of the nuclear spin in a region of an examination subject O, such as a portion of a human body that is to be examined, and who is lying on a table 23 to be moved into the magnetic resonance apparatus 5. The high degree of homogeneity in the basic magnetic field necessary for the magnetic resonance measurement (data acquisition) is defined in a typically sphere-shaped measurement volume M, in which the portion of the human body that is to be examined is placed. To support the homogeneity requirements, temporally-constant effects are eliminated by shim-plates made of ferromagnetic materials that are placed at appropriate positions. Temporally-variable effects are eliminated by shim-coils 2 and an appropriate control unit 23 for the shim-coils 2.

A cylindrically-shaped gradient coil system 3 (or alternatively, gradient field system) is incorporated in the basic field magnet 1, composed of three windings. Each winding is supplied by a corresponding amplifier Gx, Gy, and Gz, with power for generating a linear gradient field in a respective axis of a Cartesian coordinate system. The first partial winding of the gradient field system 3 generates a gradient Gx in the x-axis, the second partial winding generates a gradient Gy in the y-axis, and the third partial winding generates a gradient Gz in the z-axis. Each corresponding amplifier Gx, Gy and Gz has a digital-analog converter (DAC), controlled by a sequence controller 18 for the accurately-timed generation of gradient pulses.

A radio-frequency antenna 4 is located within the gradient field system 3, which converts the radio-frequency pulses provided by a radio-frequency power amplifier 24 into a magnetic alternating field for the excitation of the nuclei by tipping (“flipping”) the spins in the subject or the region thereof to be examined, from the alignment produced by the basic magnetic field. The radio-frequency antenna 4 is composed of one or more RF transmitting coils and one or more RF receiving coils in the form of an annular, linear, or matrix type configuration of coils. The alternating field based on the precessing nuclear spin, i.e., the nuclear spin echo signal normally produced from a pulse sequence composed of one or more radio-frequency pulses and one or more gradient pulses, is also converted by the RF receiving coils of the radio-frequency antenna 4 into a voltage (measurement signal), which is transmitted to a radio-frequency system 22 via an amplifier 7 of a radio-frequency receiver channel 8, 8′.

The radio-frequency system 22 furthermore has a transmitting channel 9, in which the radio-frequency pulses for the excitation of the magnetic nuclear resonance are generated. For this purpose, the respective radio-frequency pulses are digitally depicted in the sequence controller 18 as a series of complex numbers, based on a given pulse sequence provided by the system computer 20. This number series is sent via an input 12, in each case, as real and imaginary number components to a digital-analog converter (DAC) in the radio-frequency system 22 and from there to the transmitting channel 9. The pulse sequences are modulated in the transmitting channel 9 to a radio-frequency carrier signal, the base frequency of which corresponds to the resonance frequency of the nuclear spin in the measurement volume. The modulated pulse sequences of the RF transmitter coil are transmitted to the radio-frequency antenna 4 via an amplifier 24.

Switching from transmitting to receiving operation occurs via a transmission-receiving switch 6. The RF transmitting coil of the radio-frequency antenna 4 radiates the radio-frequency pulse for the excitation of the nuclear spin in the measurement volume M and scans the resulting echo signals via the RF receiving coils. The corresponding magnetic resonance signals obtained thereby are demodulated to an intermediate frequency in a phase sensitive manner in a first demodulator 8′ of the receiving channel of the radio-frequency system 22, and digitalized in an analog-digital converter (ADC). This signal is then demodulated to the base frequency. The demodulation to the base frequency and the separation into real and imaginary parts occurs after digitization in the spatial domain in a second demodulator 8, which emits the demodulated data via outputs 11 to an image processor 17.

In an image processor 17, an MR image is reconstructed from the measurement data obtained in this manner, which includes computation of at least one disturbance matrix and the inversion thereof, in the image processor 17. The management of the measurement data, the image data, and the control program occurs via the system computer 20. The sequence controller 18 controls the generation of the desired pulse sequences and the corresponding scanning of k-space with control programs. The sequence controller 18 controls accurately-timed switching (activation) of the gradients, the transmission of the radio-frequency pulse with a defined phase amplitude, and the reception of the magnetic resonance signals. The time base for the radio-frequency system 22 and the sequence controller 18 is provided by a synthesizer 19. The selection of appropriate control programs for the generation of an MR image, which are stored, for example, on a DVD 21, as well as other user inputs such as any suitable number N of adjacent clusters, which are to collectively cover the desired k-space, and the display of the generated MR images, occurs via a terminal 13, which includes units for enabling input entries, such as, e.g. a keyboard 15, and/or a mouse 16, and a unit for enabling a display, such as, e.g. a display screen.

The components within the dot-dash outline S are commonly called a magnetic resonance scanner, a magnetic resonance data acquisition scanner, or simply a scanner. The components within the dot-dash outline 10 are commonly called a control unit, a control device, or a control computer.

Thus, the magnetic resonance apparatus 5 as shown in FIG. 1 may include various components to facilitate the measurement, collection, and storage of MRI image data. The embodiments described herein are directed to the use of convolutional neural network (CNN) architectures to eliminate the need to perform an MRI to identify and locate scar tissue, such as cardiac scar tissue, using other non-MRI modalities. For instance, and as further discussed herein, the image data provided from a CT scan or other suitable medical imaging system may be used to generate anatomical mask data that is then input to the CNN, which has been trained with abstract anatomical mask training data extracted from of a cardiac region as discussed above (versus being trained with the images themselves) such that cardiac scar tissue, in this example, may be identified.

To do so, the embodiments described herein do not need to perform enhanced MRI scans on a particular patient for whom the cardiac scar tissue is to be identified, but the magnetic resonance apparatus 5 (or another imaging modality such as ultrasound, non-enhanced, MRI, etc.) may provide image data that is used to create a mask of, in this example, the cardiac tissue region for one or more patients in a training pool. This anatomical mask training data, which may correspond to the shape of the region of interest (e.g. the heart) may then be used to train the CNN for the classification of scar tissue within a non-MRI based image.

Thus, when used to do so, the magnetic resonance apparatus 5 may be configured to perform any suitable type of MRI scan to acquire the appropriate image data to produce abstract anatomical mask training data that is used to train the CNN. This may include, for example, a cardiac magnetic resonance imaging scan (also known as a CMR) as discussed above. Again, although the magnetic resonance apparatus 5 is shown and described herein for the purpose of obtaining the anatomical mask training data, this is one example of a medical imaging apparatus that may be used for this purpose. As discussed in further detail below, the CNN may be trained using anatomical mask training data from any suitable medical imaging source to reliably classify any suitable type of scar tissue from any suitable type of medical imaging technique to avoid the need to perform enhanced MRI scans.

The magnetic resonance apparatus 5 may include additional, fewer, or alternate components that are not depicted in FIG. 1 for purposes of brevity. For instance, the magnetic resonance apparatus 5 may alternatively include, or include in addition to the DVD 21, one or more non-transitory computer-readable data storage mediums in accordance with various embodiments of the present disclosure. Thus, the aforementioned non-transitory computer-readable media may be loaded, stored, accessed, retrieved, etc., via one or more components accessible to, integrated with, and/or in communication with the magnetic resonance apparatus 5 (e.g., network storage, external memory, etc.). For example, such data-storage mediums and associated program code may be integrated and/or accessed via the terminal 13, the control device 10 or components thereof such as the control computer 20, the image computer 17, the sequence controller 18, the RF system 22, etc.

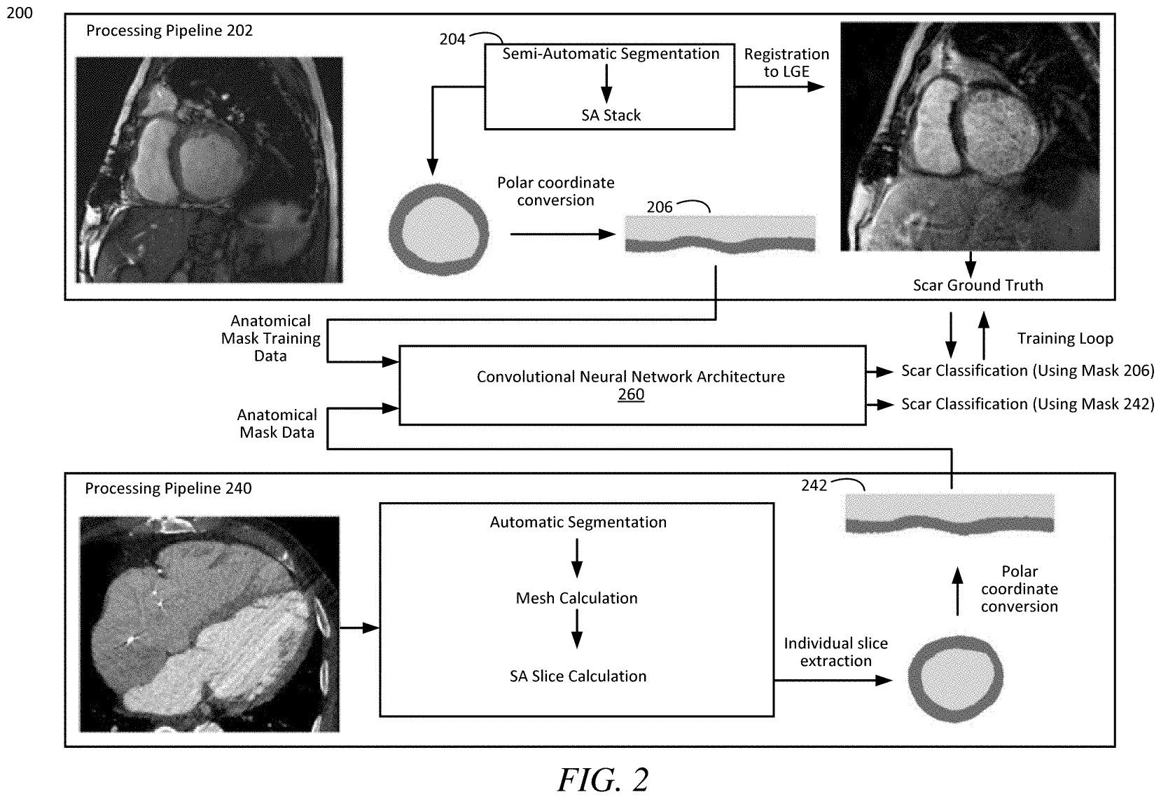

FIG. 2 illustrates a scar classification system using a convolutional neural network, in accordance with an exemplary embodiment of the present disclosure. As shown in FIG. 2, the scar classification system 200 includes processing pipelines 202, 240, and a convolution neural network (CNN) 260.

In various embodiments, the processing pipelines 202, 240 may be implemented as part of their respective imaging modalities (e.g., an MRI scanner and a CT scanner, respectively), as part of one or more separate processing components that are implemented via the scar classification system 200, or a combination of these.

For example, the processing pipeline 202 may be implemented as a portion of the magnetic resonance scanner 5 as shown in FIG. 1 (e.g., the control unit 10). To provide another example, the processing pipeline 240 may be implemented as a portion of another imaging modality, which may be a non-MRI imaging modality such as a CT scanner, for instance.

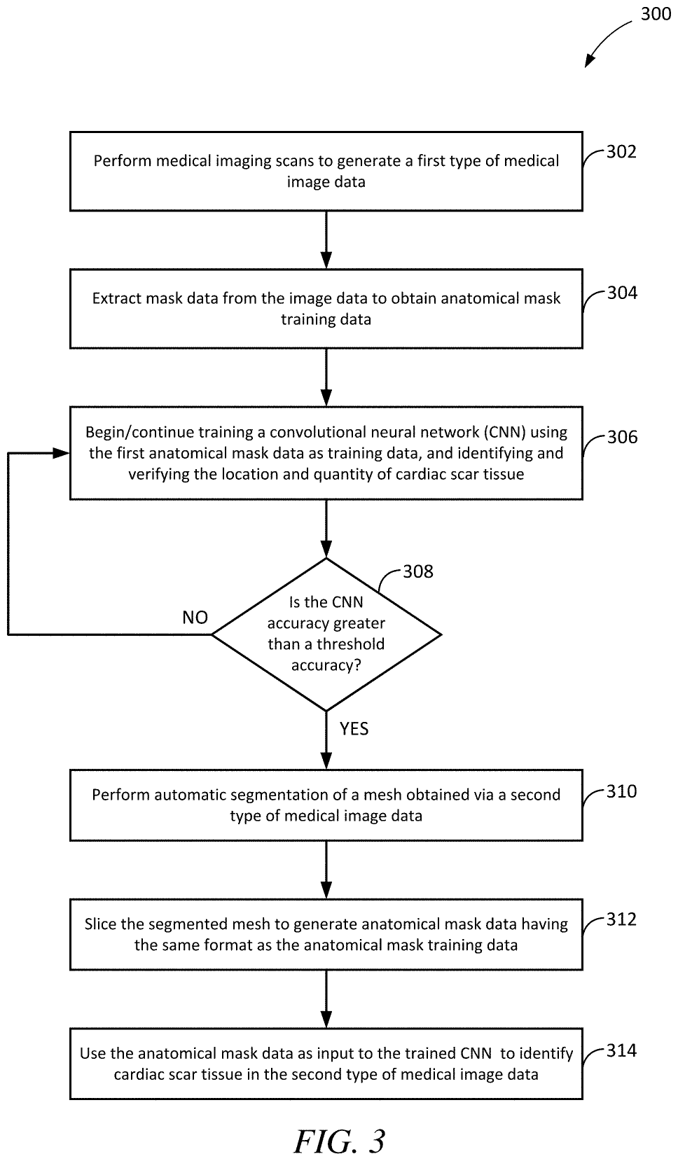

As yet another example, the processing pipelines 202 and/or 240 may be implemented as one or more suitable processing components, software components (e.g. image processing algorithms), or a combination of hardware and software components. These components may be separate from their respective imaging modalities. In such a case, the processing pipelines 202 and/or 240 may access, load, and/or otherwise retrieve their respective image data in any suitable manner, such as via communication with their respective imaging modalities, via automatic loading or retrieval of the image data, etc. Furthermore, the processing pipelines 202, 240 and/or the CNN 260 may be integrated as part of a common system and/or controlled via a common system. In such a case, the various components of the scar classification system 200 may be controlled via one or more processors (which may be integrated as constituent processor components of the processing pipelines 202, 240, and/or the CNN 260 or as separate processing components) and execute instructions stored on a non-transitory computer-readable medium. The method as shown and discussed further below with respect to FIG. 3 may also be implemented via the scar classification system 200 and/or via the execution of instructions stored in such a non-transitory computer-readable medium, which is not shown in the Figures for purposes of brevity.

In any event, the processing pipelines 202, 240 are each configured to generate specific data sets that are used by the CNN 260, as shown in FIG. 2. With respect to the processing pipeline 202, the anatomical mask training data 206 may include data that is used as a model input to the algorithmic model executed by the CNN 260. For instance, continuing the previous example in which the processing pipeline 202 may be implemented in accordance with a CMR scan, the processing pipeline 202 may generate the anatomical mask training data 206 as an aggregation of masks extracted from a scanned region of multiple patients, and may correspond to a specific anatomical shape, such as a patient's heart in this example.

To do so, the processing pipeline 202 may perform image processing tasks such as semi-automatic segmentation and a short axis (SA) stack acquisition of acquired CMR images. SA stack acquisition is a known technique that typically provides several parallel slices of multiple cardiac phases, which is commonly used in the assessment of ventricular function. In an embodiment, the processing pipeline 202 may further utilize the obtained SA stack data to perform polar coordinate conversion, thus extracting the left ventricle wall masks from the MRI scans to provide the abstract anatomical mask training data 206 in a form which has removed any location variance introduced by the MRI operator when defining the imaging planes. In other words, the anatomical mask training data 206 includes left ventricle wall masks that provide the model input data included in the first type of medical imaging data for the training loop.

Further, and as shown in FIG. 2, the processing pipeline 202 is configured to register the SA stacks with the late gadolinium enhanced (LGE) data to provide image data that includes the “ground truth,” or the empirical data associated with the result of the contrast scan (or other suitable scan used for verifying the location and quantity of scar tissue), which provides an accurate and reliable result identifying the actual cardiac scar tissue in the CMR images that is used as part of the training loop for the CNN 260, as further discussed below. In other words, the ground truth data defines a correct determination of the location and quantity of cardiac scar tissue from images in which the anatomical mask training data was extracted, which is then used to train the CNN.

For instance, although the processing pipeline 202 outputs the cardiac scar ground truth data, this data is not input into the algorithmic model implemented via the CNN 260 to identify the cardiac scar tissue. Instead, the ground truth data may be used as part of verification data in a training loop to train the CNN 260. In particular, the CNN 260 uses the anatomical mask training data 206, which includes the extracted left ventricle wall mask data as the model input data, and attempts to identify a location and quantity of cardiac scar tissue included in the medical imaging data (MRI image data in this example). In other words, the CNN 260 “infers” the location of the cardiac scar tissue within the MRI images using the anatomical mask training data 206. This determination may then be verified with the ground truth data or scar data as shown in FIG. 2, and repeated any suitable number of iterations and for any suitable number of anatomical mask training data generated via the processing pipeline 202 until a desired accuracy is obtained.

In other words, the CNN 260 is trained using the anatomical mask data 260 as a model input, as the anatomical mask training data 206 shows cardiac wall thickness and shape, and can be extracted using various imaging modalities other than MRI scans. Thus, the anatomical mask training data 206 is used to train the CNN model by iteratively verifying the inferred location and quantity of cardiac scar tissue output by the CNN model with the location and quantity of cardiac scar tissue determined via a reliable scar identification technique (e.g., a LGE cardiac MRI scan or other suitable medical imaging techniques) known to provide reliable results) as part of a CNN training loop. Doing so advantageously allows for the accurate identification of cardiac scar tissue location and quantity using other less costly or more convenient medical imaging modalities once the CNN 260 is trained in this way.

For instance, and continuing the example in which the processing pipeline 240 operates in accordance with a CT scanning imaging modality, once the algorithmic model to the CNN 260 is trained in a manner that accurately identifies cardiac scar tissue from the anatomical mask training data 260 (e.g. greater than a desired threshold accuracy) the processing pipeline 240 may then execute image-processing tasks in accordance with acquired CT scan images to subsequently provide the anatomical mask data 242 to the convolutional neural network 260. This may include, as shown in FIG. 2, the application of automatic segmentation, mesh calculations, and SA slice calculations. For example, for a cardiac scan, the processing pipeline 240 may perform automatic segmentation of the volume of cardiac tissue, perform a volumetric mesh calculation, and then obtain, from this calculated volumetric mesh, SA slices. These SA slices may then be used to extract the wall thickness and shape of the cardiac tissue to generate the anatomical mask data 242, which is provided as an input to the trained convolutional neural network 260 to classify the scar tissue, i.e. to determine the location and quantity of the cardiac scar tissue based upon the data received, which is the anatomical mask data 242. The process of performing automatic segmentation of a particular volume, creating a mesh, and performing SA slice calculations are known techniques in the field of CT scanning as well as other types of medical imaging modalities, and thus additional details of these image processing steps are not further discussed herein.

In an embodiment, the processing pipeline 240 is configured to generate the anatomical mask data 242 having the same mask format as the anatomical mask training data 206 or, more specifically, the portion or entirety of the anatomical mask training data 206 that was used as the model input to train the CNN. For instance, the polar coordinate conversion applied to the individual slice extractions may be performed in a predetermined manner based upon the known data format of the portion of the anatomical mask training data 206 that was used as the model input to train the CNN. This yields a resulting mask format of the anatomical mask data 242 that matches that of the data associated with the anatomical mask training data 206 used to train the CNN 260. In doing so, it is assured that the CNN 260 can reliably recognize cardiac scar tissue from the input anatomical mask data 242, as the CNN 260 has already been trained to reliably identify scar tissue locations and quantity in a similar manner, albeit with mask data obtained via a different type of medical imaging modality.

Therefore, the use of the automatic segmentation tool, which is in this case based upon acquired CT images but may be adapted in accordance with any suitable medical imaging modality, may be particularly useful to generate the image mask of the cardiac anatomy (e.g. showing endocardium and epicardium walls). In other words, the anatomical mask 242, although obtained via a non-MRI imaging modality, advantageously represents an abstract anatomical mask that is similar to the mask data used to train the CNN 260.

In various embodiments, the CNN 260 may have any suitable type of architecture and be trained in accordance with any suitable techniques using the training loop as shown and discussed above with reference to FIG. 2. For instance, the convolutional neural network may have an input layer that is configured to receive the anatomical mask data 242 as one or more images, multiple hidden layers (e.g. Cony, ReLu, and Crop pooling), which function to filter, rectify, and downsample the processed data, as well as an output layer that is configured to classify pixels in the image data as cardiac scar tissue, as non-cardiac scar tissue, or as any other suitable type of tissue in accordance with the training of the CNN 260. The model used by the CNN 260 may include, for example, any suitable type of CNN-based algorithm configured to recognize and/or classify components of image data once trained as discussed herein. For instance, the training loop as shown and discussed herein with reference to FIG. 2 may form part of a “backprop” step that is typically used for CNN training using a comparison of outputs to a desired or known result.

Moreover, embodiments include the CNN 260 being trained using any suitable number and/or type of scaling and mask production, which may include simulated anatomical mask training data or training data obtained via any suitable medical imaging source. When trained, embodiments include the CNN 260 predicting scar tissue using CT imaging or other suitable medical imaging modalities. Again, any suitable type of medical imaging modalities in which meshes can be generated can work in accordance with their own processing pipelines using the same model as described herein.

To summarize, embodiments of the scar classification system 200 facilitate the training of the CNN 260 with anatomical mask data (e.g. the shape of the heart) instead of image data itself. Thus, the embodiments as discussed herein may derive the anatomical mask data that is used to train the network via one modality (e.g. MRI) and, once trained, the trained CNN 260 may be used to predict results for anatomical mask data obtained via another imaging modality (e.g. CT data). Moreover, because the input to the CNN 260 is a mask of the heart anatomy, as opposed to the images themselves, the CNN 260 may be trained in accordance with a general scar detection algorithm, which can be used for MRI, CT, or any other suitable anatomical imaging method, depending upon scanner availability or what additional data the clinician requires.

Since the embodiments described herein use an anatomical mask derived from imaging modalities, it is not limited to only being trained in accordance with using enhanced cardiac LGE MRI described herein. The same principles described herein may also apply to any imaging modality in which the wall of a heart structure may be derived together with scar locations for training purposes. For example, PET or scar tissue derived from ultrasound could potentially be used as alternate training methods. The resulting model can thus be used on any modality in which the heart wall can be segmented to produce similar anatomical abstractions. Such modalities include, for instance, ultrasound, non-enhanced MRI scans, etc.

In other words, the present disclosure preferably provides use of abstract anatomical masks as input to make cardiac scar detection modality independent. As an example, CT scans may be used to accurately identify cardiac scar tissue via the application of a properly-trained CNN when only MRI data is available as a ground truth. Thus, by leveraging the use of a CNN as discussed herein that is trained using anatomical mask data, the embodiments described herein facilitate automatic cardiac scar detection without contrast enhanced scanning protocols such as MRI or PET.

Advantageously for healthcare providers, each CT scan is cheaper and faster than an equivalent MRI scan. Also, being able to detect cardiac scar tissue and avoid the use of an MRI improves efficiency and lowers cost. By providing cardiac scar detection using CT data, as one example, which conventionally is not provided in general clinical practice, a case may also be made for the use of cardiac CT over MRI in some cases.

FIG. 3 is an example flow, in accordance with an exemplary embodiment of the present disclosure. With reference to FIG. 3, the flow 300 may be a computer-implemented method executed by and/or otherwise associated with one or more processors and/or storage devices. These processors and/or storage devices may be, for instance, associated with a processing pipeline of a particular imaging modality, a convolutional neural network, and/or a modality-independent processing system, such as those described herein with reference to the scar classification system 200 as shown in FIG. 2, for example. Moreover, in an embodiment, flow 300 may be performed via one or more processors executing instructions stored on a suitable storage medium (e.g., a non-transitory computer-readable storage medium). In an embodiment, the flow 300 may describe an overall operation to identify scar tissue using one imaging modality with a CNN that has been trained with mask data extracted via another imaging modality. Embodiments may include alternate or additional steps that are not shown in FIG. 3 for purposes of brevity.

Flow 300 may begin when one or more processors perform (block 302) medical imaging scans in accordance with a particular imaging modality. This may include, for example, the use of CMR to collect CMR imaging data for the cardiac region of a patient, as discussed herein with respect to FIG. 2.

Flow 300 may further include one or more processors extracting (block 304) anatomical mask training data and ventricle wall mask data from the obtained (block 302) image data. This may include, for example, the generation of the anatomical mask training data 206, as discussed herein with respect to FIG. 2.

Flow 300 may further include one or more processors training (block 306) a CNN using the anatomical mask training data as at least a portion of model input data that is utilized by the CNN. This may include, for example, training the CNN 260 using the anatomical mask training data 206, as discussed herein with respect to FIG. 2, by verifying the results of the inferred location and quantity of cardiac scar tissue output by the CNN algorithmic model with a known reliable imaging modality (e.g., LGE CMR).

Flow 300 may further include one or more processors continuing (block 308) the training process by iteratively verifying the inferred location and quantity of cardiac scar tissue output by the CNN algorithmic model with a known reliable imaging modality. Once a desired threshold accuracy if obtained (YES), then the method flow 300 may continue. Otherwise (NO), the method flow 300 may revert to continuing the training (block 306) process.

Flow 300 may further include one or more processors performing (block 310) automatic segmentation of a mesh using another type of medical imaging data, which is different than the generated (block 302(medical imaging data used to extract (block 304) the anatomical mask training data, to provide segmented mesh data. This may include, for example, the automatic segmentation of CT scan data via any suitable tools or techniques (including known techniques), as discussed herein with respect to FIG. 2.

Flow 300 may further include one or more processors performing (block 312) image slicing of the segmented mesh data to generate anatomical mask data having a mask format that is the same as that of the anatomical mask training data. This may include, for example, performing SA slice calculations of CT scan data via any suitable tools or techniques (including known techniques), as discussed herein with respect to FIG. 2.

Flow 300 may further include one or more processors using the anatomical mask data to identify (block 314), via the trained CNN, a location and quantity of scar tissue within the second type of medical imaging data. This may include, for example, identifying a location and/or quantity of cardiac scar tissue from CT scan data, as discussed herein with respect to FIG. 2.

Although the present disclosure has been illustrated and described in detail with the preferred exemplary embodiments, the disclosure is not restricted by the examples given, and other variations can be derived therefrom by a person skilled in the art without departing from the protective scope of the disclosure. Although modifications and changes may be suggested by those skilled in the art, it is the intention to embody all changes and modifications as reasonably and properly come within the scope of their contribution to the art.

It is also pointed out for the sake of completeness that the use of the indefinite articles “a” or “an” does not exclude the possibility that the features in question may also be present more than once. Similarly, the term “unit” does not rule out the possibility that the same consists of a plurality of components which, where necessary, may also be distributed in space.

The claims described herein and the following description in each case contain additional advantages and developments of the embodiments as described herein. In various embodiments, the claims of one claims category can, at the same time, be developed analogously to the claims of a different claims category and the parts of the description pertaining thereto. Furthermore, the various features of different exemplary embodiments and claims may also be combined to create new exemplary embodiments without departing from the spirit and scope of the disclosure.

### REFERENCES

The following references are cited throughout this disclosure as applicable to provide additional clarity, particularly with regards to terminology. These citations are made by way of example and ease of explanation and not by way of limitation.

Citations to the following references are made throughout the application using a matching bracketed number, e.g., [1].

[1] Esposito, A., Palmisano, A., Antunes, S., Maccabelli, G., Colantoni, C., Rancoita, P. M. V., Del Maschio, A. (2016). Cardiac CT with Delayed Enhancement in the Characterization of Ventricular Tachycardia Structural Substrate. JACC: Cardiovascular Imaging, 9(7), 822-832.

[2] Gerber, B. L., Belge, B., Legros, G. J., Lim, P., Poncelet, A., Pasquet, A., Vanoverschelde, J.-L. J. (2006). Characterization of Acute and Chronic Myocardial Infarcts by Multidetector Computed Tomography: Comparison with Contrast-Enhanced Magnetic Resonance. Circulation, 113(6), 823-833.

[3] Kali, A., Cokic, I., Tang, R. L. Q., Yang, H. J., Sharif, B., Marb ìan, E., Li, D., Berman, D. S., Dharmakumar, R.: Determination of location, size, and transmurality of chronic myocardial infarction without exogenous contrast media by using cardiac magnetic resonance imaging at 3 T. Circulation. Cardiovascular imaging 7(3), 471-81 (5 2014).

[4] Flett, A. S., Hasleton, J., Cook, C., Hausenloy, D., Quarta, G., Ariti, C., Moon, J. C. (2011). Evaluation of Techniques for the Quantification of Myocardial Scar of Differing Etiology Using Cardiac Magnetic Resonance. JACC: Cardiovascular Imaging, 4(2), 150-156.

[5] Crean, A., Khan, S. N., Davies, L. C., Coulden, R., & Dutka, D. P. (2009). Assessment of Myocardial Scar; Comparison between F-FDG PET, CMR and Tc-Sestamibi. Clinical Medicine. Cardiology, 3, 69-76.

[6] Cedilnik, N., Duchateau, J., Dubois, R., Jais, P., Cochet, H., Sermesant, M.: VT Scan: Towards an Efficient Pipeline from Computed Tomography Images to Ventricular Tachycardia Ablation. In: Functional Imaging and Modelling of the Heart. pp. 271-279. Springer, Cham (2017). https://doi.org/10.1007/978-3-319-59448-4 26.

[7] Rasmussen, S., Corya, B. C., Feigenbaum, H., & Knoebel, S. B. (1978). Detection of myocardial scar tissue by M-mode echocardiography. Circulation, 57(2), 230-7.

What is claimed is:

1. A method for detecting scar tissue within image data, the method comprising:
extracting, via one or more processors, anatomical mask training data from a first type of medical imaging data;
training, via one or more processors, a convolutional neural network (CNN) using the anatomical mask training data as at least a portion of model input data that is utilized by the CNN;
performing, via one or more processors, automatic segmentation of a mesh from a second type of medical imaging data, which is different than the first type of medical imaging data, to provide segmented mesh data;
performing, via one or more processors, image slicing of the segmented mesh data to generate anatomical mask data having a mask format that is the same as that of the anatomical mask training data; and
identifying, via the trained CNN, a location of scar tissue within the second type of medical imaging data using the CNN input data.

2. The method of claim 1, wherein the scar tissue within the second type of medical imaging data is cardiac scar tissue.

3. The method of claim 1, wherein the act of extracting the anatomical mask training data includes extracting each one of the set of left ventricle wall masks as a plurality of slices extracted from each one of a set of different patients in a training pool.

4. The method of claim 3, further comprising:
outputting, using the first type of medical imaging data, scar data representative of an expected location and quantity of cardiac scar tissue included in the first type of medical imaging data.

5. The method of claim 1, wherein the first type of medical imaging data is obtained via a cardiac magnetic resonance imaging scan, and
wherein the second type of medical imaging data is computerized tomography (CT) image data obtained via a CT scan.

6. The method of claim 1, wherein the act of training the CNN comprises:
determining a location and quantity of cardiac scar tissue included in the first type of medical imaging data using the anatomical mask training data; and
iteratively verifying the location and quantity of the cardiac scar tissue with a result determined via a late gadolinium enhanced (LGE) magnetic resonance imaging (MRI) scan as part of a CNN training loop.

7. A system for detecting cardiac scar tissue within image data, the system comprising:
a first processing pipeline configured to extract anatomical mask training data from a first type of medical imaging data;
a convolutional neural network (CNN) configured to be trained using the anatomical mask training data as at least a portion of model input data that is utilized by the CNN; and
a second processing pipeline configured to (i) perform automatic segmentation of a mesh from a second type of medical imaging data, which is different than the first type of medical imaging data, to provide segmented mesh data, and (ii) perform image slicing of the segmented mesh data to generate anatomical mask data having a mask format that is the same as that of the anatomical mask training data,
wherein the CNN is further configured, once trained, to identify a location of scar tissue within the second type of medical imaging data using the CNN input data.

8. The system of claim 7, wherein the scar tissue within the second type of medical imaging data is cardiac scar tissue.

9. The system of claim 7, wherein the first processing pipeline is configured to extract the anatomical mask training data including each one of the set of left ventricle wall masks as a plurality of slices extracted from each one of a set of different patients in a training pool.

10. The system of claim 9, wherein the first processing pipeline is configured to output scar data representative of an expected location and quantity of cardiac scar tissue included in the first type of medical imaging data using the first type of medical imaging data.

11. The system of claim 7, wherein the first type of medical imaging data is obtained via a cardiac magnetic resonance imaging scan, and
wherein the second type of medical imaging data is computerized tomography (CT) image data obtained via a CT scan.

12. The system of claim 7, wherein the CNN is configured to be trained by:
determining a location and quantity of cardiac scar tissue included in the first type of medical imaging data using the anatomical mask training data; and
iteratively verifying the location and quantity of the cardiac scar tissue with a result determined via a late gadolinium enhanced (LGE) magnetic resonance imaging (MRI) scan as part of a CNN training loop.

13. A non-transitory computer readable medium having one or more instructions stored thereon that, when executed by a processing system, cause the processing system to:
extract anatomical mask training data from a first type of medical imaging data;
train a convolutional neural network (CNN) using the anatomical mask training data as at least a portion of model input data that is utilized by the CNN;
perform automatic segmentation of a mesh from a second type of medical imaging data, which is different than the first type of medical imaging data, to provide segmented mesh data;
perform image slicing of the segmented mesh data to generate anatomical mask data having a mask format that is the same as that of the anatomical mask training data; and
identify a location of scar tissue within the second type of medical imaging data using the CNN input data.

14. The non-transitory computer readable medium as claimed in claim 13, wherein the scar tissue within the second type of medical imaging data is cardiac scar tissue.

15. The non-transitory computer readable medium as claimed in claim 13, wherein the anatomical mask training data is extracted to include each one of the set of left ventricle wall masks as a plurality of slices extracted from each one of a set of different patients in a training pool.

16. The non-transitory computer readable medium as claimed in claim 15, further including instructions that, when executed by the processing system, cause the processing system to output, using the first type of medical imaging data, scar data representative of an expected location and quantity of cardiac scar tissue included in the first type of medical imaging data.

17. The non-transitory computer readable medium as claimed in claim 13, wherein the first type of medical imaging data is obtained via a cardiac magnetic resonance imaging scan, and
wherein the second type of medical imaging data is computerized tomography CT image data obtained via a CT scan.

18. The non-transitory computer readable medium as claimed in claim 13, further including instructions that, when executed by the processing system, cause the CNN to be trained by:
determining a location and quantity of cardiac scar tissue included in the first type of medical imaging data using the anatomical mask training data; and
iteratively verifying the location and quantity of the cardiac scar tissue with a result determined via a late gadolinium enhanced (LGE) magnetic resonance imaging (MRI) scan as part of a CNN training loop.
